(19) United States
(12) Patent Application Publication
(10) Pub. No.: US 2020/0289695 A1
(43) Pub. Date: Sep. 17, 2020

(54) FRAGRANCE DISPERSING DEVICE

(71) Applicant: Dustin McKinney, Luck, WI (US)

(72) Inventor: Dustin McKinney, Luck, WI (US)

(21) Appl. No.: 16/351,990

(22) Filed: Mar. 13, 2019

(51) Int. Cl. A61L 9/12; A61L 9/04

(52) U.S. Cl. CPC A61L 9/122; A61L 2209/15; A61L 9/048

(57) ABSTRACT

A device for dispersing a fragrance by means of an electrical fan; the device includes a first half of a housing, a second half of a housing, and a fragrance-composition. The device is attachable to the electrical fan and is capable of dispensing scent when installed in front of the electrical fan.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of fragrance dispersers and more specifically relates to a device for dispersing fragrance that is attachable to a fan.

### 2. Description of Related Art

Keeping homes, offices, and the like, smelling fresh and clean can be difficult. People may use candles or air fresheners to keep fresh-smelling scents present; however, some of these items may not last long and can be expensive to purchase. Others may be unsafe and catch fire. Some scent dispensers require an electrical outlet in order to activate, and some rooms may be unable to support the device. A suitable solution is desired.

U.S. Pub. No. 2015/0016995 to Nathan A. Scolari relates to an apparatus for distributing a fragrance using a fan. The described apparatus for distributing a fragrance using a fan includes a housing having openings defined in the housing; a fragrance reservoir included in the housing for receiving a fragrance medium containing a fragrance; a cover operably associated with the opening for restricting airflow into the fragrance reservoir; and, a cover actuator operably connected to the cover having an open position allowing airflow into and out of the fragrance reservoir for dispensing a fragrance and a closed position for restricting airflow into and out of the fragrance reservoir.

### BRIEF SUMMARY OF THE INVENTION

In view of the foregoing disadvantages inherent in the known fragrance dispersers art, the present disclosure provides a novel fragrance dispersing device. The general purpose of the present disclosure, which will be described subsequently in greater detail, is to provide users with a plastic cap capable of dispensing scent when installed in front of a fan.

A device is disclosed herein. The device includes a first half of a housing which may include a first front surface and a first rear surface. The front surface may have a first fastener thereon, and the rear surface may have a fan attachment-means thereon. The fan attachment-means may be configured to attach the first half to the fan blade shield. Similarly, a second half of the housing may be provided. The second half may include a second front surface and a second rear surface. The second rear surface may have a second fastener thereon configured to removably attach the second half to the first half.

The second half may further include an inner-capacity and a fragrance-outlet. A fragrance-composition may be housed within the inner-capacity of the second-half and may include the fragrance. The fragrance may be configured to disperse through the fragrance-outlet when the electrical fan may be in an on-state.

A method of using the device is also disclosed herein. The method of using the device may comprise the steps of: providing the device as above; attaching the first half of the housing to the fan blade shield via the fan attachment-means; attaching the second half of the housing to the first half of the housing via the first fastener and the second fastener; and turning the electrical fan to the on-state to disperse the fragrance through expelled air.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a fragrance dispersing device, constructed and operative according to the teachings of the present disclosure.

FIG. 1 is a side-front perspective view of the device during an ‘in-use’ condition, according to an embodiment of the disclosure.

FIG. 2 is a side perspective view of the device of FIG. 1, according to an embodiment of the present disclosure.

FIG. 3 is a side-front perspective view of the device of FIG. 1, according to an embodiment of the present disclosure.

FIG. 4 is a side-rear perspective view of the device of FIG. 1, according to an embodiment of the present disclosure.

FIG. 5 is a flow diagram illustrating a method of use for the device, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a fragrance disperser and more particularly to a fragrance dispersing device as used to improve dispersing of fragrance via means that is attachable to a fan.

Generally disclosed is a device which allows different types of scent capsules to be slid in and out, allowing users to customize the scents in their homes, offices, and the like. This keeps any room smelling fresh, clean, and sanitary at all times. The device offers easy manual activation by pressing the cap once to activate the scent dispenser and a second time to turn it off. The device may eliminate the need to burn candles or spend extensive amounts of money on other, typically ineffective, household scent dispensers.

The device may include a cap unit that is capable of dispensing fresh-smelling fragrances. The cap may be installed on the front of an electrical fan and be activated by turning the cap. There can be two holes for the cap attachment to slide into the center portion of the fan. The caps can be interchangeable as needed, offering a way to obtain different types of fragrances within a specific room of a home or office. Users may activate the device by turning the cap to the right and shut off by turning the cap back to the left. The device may be constructed using plastic and other suitable materials. Further, the device may be available in various sizes in order to accommodate all user needs and preferences. Exact size, measurement, construction, and design specifications may vary upon manufacturing.

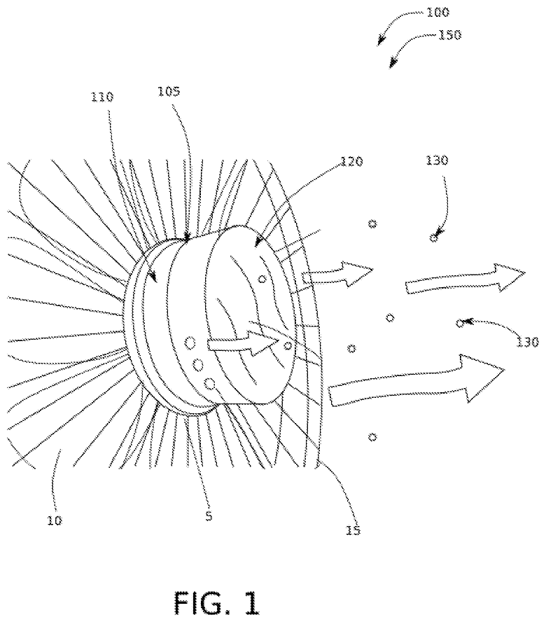

Referring now more specifically to the drawings by numerals of reference, there is shown in FIGS. 1-5, various views of a device 100.

FIG. 1 shows a device 100 during an ‘in-use’ condition 150, according to an embodiment of the present disclosure. As illustrated, the device 100 may include a first half 110 of a housing 105, a second half 120 of the housing 105 and a fragrance-composition 130. The device 100 may be used for dispersing a fragrance by means of an electrical fan 5, the electrical fan 5 including fan blades 10 and a fan blade shield 15 for covering the fan blades 10.

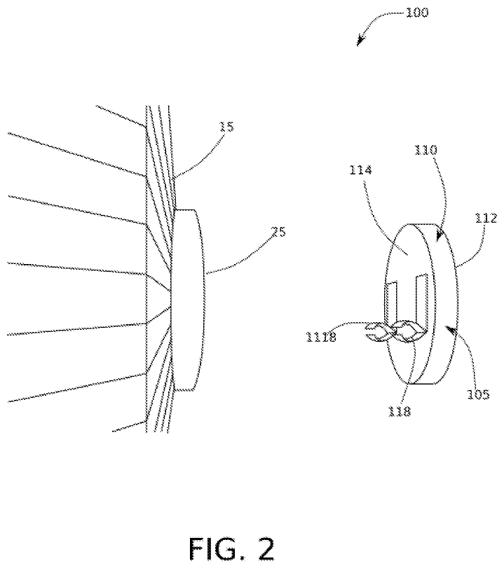

FIG. 2 shows a side perspective view of the device 100 of FIG. 1, according to an embodiment of the present disclosure. The first half 110 of the housing 105 may include a first front surface 112 and a first rear surface 114. The first rear surface 114 may have a fan attachment-means 118 thereon. The fan attachment-means 118 may be configured to attach the first half 110 to the fan blade shield 15.

In one embodiment, the fan attachment-means 118 may include at least one hook. Preferably, the at least one hook may be two hooks. A first hook of the two hooks may be located on a first side of the first rear-surface of the first half 110, and a second hook of the two hooks may be located opposite the first hook on a second side of the first rear-surface of the first half 110. In this embodiment, the two hooks may easily hook onto, and secure the first half 110 to, bars on the fan blade shield 15. Other fan attachment-means 118 are contemplated, however. For example, the fan attachment-means 118 may include hook and loop fastener. Other examples include clips, clamps, adhesives, etc. The first half 110 may be removably attached to the fan blade shield 15 in one embodiment, or permanently attached in another embodiment.

As shown, the fan blade shield 15 may include a shield center 25 and the first half 110 may be sized to attach to the shield center 25. The fan blade shield 15 may be circular in shape, and likewise, the first half 110 may be circular in shape. Further, the shield center 25 and the first half 110 may be equal in diameter. In another embodiment, the shield center 25 may be larger in diameter to the first half 110, to allow the first half 110 to fit comfortably within the shield center 25. In one embodiment, the fan attachment-means may include an aperture (not illustrated) that is able to mate with the shield center 25.

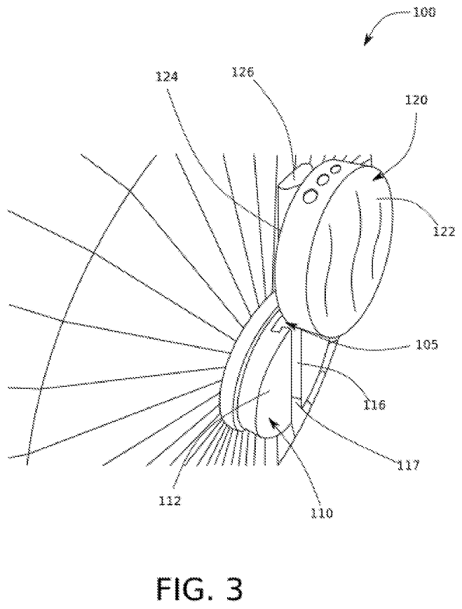

FIG. 3 shows a side-front perspective view of the device 100 of FIG. 1, according to an embodiment of the present disclosure. The first half 110 of the housing 105 may include a first front surface 112. Preferably, the first front surface 112 may have a first fastener 116 thereon. The second half 120 of the housing 105 may include a second front surface 122 and a second rear surface 124. The second rear surface 124 may have a second fastener 126 thereon which may be configured to removably attach the second half 120 to the first half 110. As shown, the second fastener 126 may protrude from the second rear surface 124 of the second half 120, and the first fastener 116 may include an aperture 117 configured to slidably receive the second fastener 126. Similarly to the first half 110, the second half 120 may be circular in shape. Preferably, the first half 110 and the second half 120 may be equal in size. However, it is contemplated that the first half 110 and the second half 120 may differ in size.

The first half 110 and the second half 120 may be made from a durable material configured to withstand cooler temperatures dispersed by the electrical fan 5. In one embodiment, the durable material may be a plastic material. Other materials include metal, wood, or other suitably equivalent materials, etc. Further, as shown, the second front surface 122 of the second half 120 may include a functional decorative pattern thereon. The functional decorative pattern may further bear indicia.

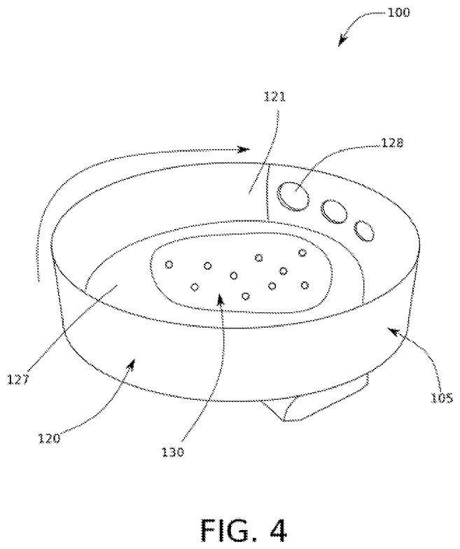

FIG. 4 shows a side-rear perspective view of the device 100 of FIG. 1, according to an embodiment of the present disclosure. The second half 120 may further include an inner-capacity 127 and a fragrance-outlet 128. Preferably, the fragrance-outlet 128 may include for functional use an open-state and a closed-state. Further, the second half 120 may include an outlet-cover 121 configured to prevent airflow into the inner-capacity 127 and further prevent the fragrance from being dispersed through the fragrance-outlet 128 when in the closed-state. A user may twist the second half 120 to cover the fragrance-outlet 128 with the outlet-cover 121.

The fragrance-composition 130 may be housed within the inner-capacity 127 of the second half 120. The fragrance-composition 130 may include the fragrance configured to disperse through the fragrance-outlet 128 when the electrical fan 5 is an on-state. In one embodiment, the fragrance-composition 130 may be a gel. In other examples, the fragrance-composition 130 may be a liquid or a solid. The fragrance-composition 130 may contain at least 70% of the fragrance. The fragrance is contemplated to be manufactured in a plurality of scents.

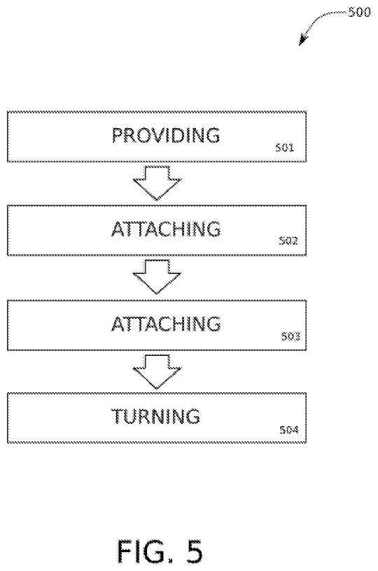

FIG. 5 is a flow diagram illustrating a method of using a device for dispersing a fragrance 500, according to an embodiment of the present disclosure. As illustrated, the method of using a device for dispersing a fragrance 500 may include the steps of: providing 501 the device as above; attaching 502 the first half of the housing to the fan blade shield via the fan attachment-means; attaching 503 the second half of the housing to the first half of the housing via the first fastener and the second fastener; and turning 504 the electrical fan to the on-state to disperse the fragrance through expelled air.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for use of device 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for dispersing a fragrance by means of an electrical fan, the electrical fan including fan blades and a fan blade shield for covering the fan blades, the device comprising:
a first half of a housing, the first half including a first front surface and a first rear surface, the first front surface having a first fastener thereon, and the rear surface having a fan attachment-means thereon, the fan attachment-means configured to attach the first half to the fan blade shield;
a second half of the housing, the second half including a second front surface and a second rear surface, the second rear surface having a second fastener thereon, the second fastener configured to removably attach the second half to the first half, the second half further including an inner-capacity and a fragrance-outlet; and
a fragrance-composition housed within the inner-capacity of the second-half, the fragrance-composition including the fragrance, the fragrance being configured to disperse through the fragrance-outlet when the electrical fan is an on-state.

2. The device of claim 1, wherein the fan blade shield includes a shield center, and wherein the first half is sized to attach to the shield center.

3. The device of claim 1, wherein the first half is circular in shape.

4. The device of claim 3, wherein the shield center and the first half are equal in diameter.

5. The device of claim 1, wherein the shield center is larger in diameter to the first half.

6. The device of claim 1, wherein the second half is circular in shape.

7. The device of claim 6, wherein the first half and the second half are equal in size.

8. The device of claim 1, wherein the fan attachment-means is at least one hook.

9. The device of claim 8, wherein the at least one hook is two hooks.

10. The device of claim 1, wherein the fan attachment-means includes hook and loop fastener.

11. The device of claim 1, wherein the fragrance-outlet has for functional use an open-state and a closed-state.

12. The device of claim 11, wherein the second half includes an outlet-cover configured to prevent airflow into the inner-capacity and further prevent the fragrance from being dispersed through the fragrance-outlet when in the closed-state.

13. The device of claim 1, wherein the second fastener protrudes from the second rear surface of the second half.

14. The device of claim 13, wherein the first fastener includes an aperture configured to slidably receive the second fastener.

15. The device of claim 1, wherein the fragrance-composition is a gel.

16. The device of claim 1, wherein the fragrance-composition is a liquid.

17. The device of claim 1, wherein the fragrance-composition is a solid.

18. The device of claim 1, wherein the second front surface includes a functional decorative pattern thereon.

19. A device for dispersing a fragrance by means of an electrical fan, the electrical fan including fan blades and a fan blade shield for covering the fan blades, the device comprising:
a first half of a housing, the first half including a first front surface and a first rear surface, the front surface having a first fastener thereon, and the rear surface having a fan attachment-means thereon, the fan attachment-means configured to attach the first half to the fan blade shield;
a second half of the housing, the second half including a second front surface and a second rear surface, the second rear surface having a second fastener thereon, the second fastener configured to removably attach the second half to the first half, the second half further including an inner-capacity and a fragrance-outlet; and
a fragrance-composition housed within the inner-capacity of the second-half, the fragrance-composition including the fragrance, the fragrance being configured to disperse through the fragrance-outlet when the electrical fan is an on-state;
wherein the fan blade shield includes a shield center, and wherein the first half is sized to attach to the shield center;
wherein the first half is circular in shape;
wherein the shield center and the first half are equal in diameter;
wherein the shield center is larger in diameter to the first half;
wherein the second half is circular in shape;
wherein the first half and the second half are equal in size;
wherein the fan attachment-means is at least one hook;
wherein the fan attachment-means includes hook and loop fastener;
wherein the fragrance-outlet has for functional use an open-state and a closed-state;
wherein the second half includes an outlet-cover configured to prevent airflow into the inner-capacity and further prevent the fragrance from being dispersed through the fragrance-outlet when in the closed-state;
wherein the second fastener protrudes from the second rear surface of the second half; and
wherein the first fastener includes an aperture configured to slidably receive the second fastener.

20. A method of using a device for dispersing a fragrance by means of an electrical fan, the electrical fan including fan blades and a fan blade shield for covering the fan blades, the method comprising:
providing the device for dispersing a fragrance by means of an electrical fan, the device including:
a first half of a housing, the first half including a first front surface and a first rear surface, the front surface having a first fastener thereon, and the rear surface having a fan attachment-means thereon, the fan attachment-means configured to attach the first half to the fan blade shield;
a second half of the housing, the second half including a second front surface and a second rear surface, the second rear surface having a second fastener thereon, the second fastener configured to removably attach the second half to the first half, the second half further including an inner-capacity and a fragrance-outlet; and
a fragrance-composition housed within the inner-capacity of the second-half, the fragrance-composition including the fragrance, the fragrance being configured to disperse through the fragrance-outlet when the electrical fan is an on-state;
attaching the first half of the housing to the fan blade shield via the fan attachment-means;
attaching the second half of the housing to the first half of the housing via the first fastener and the second fastener; and
turning the electrical fan to the on-state to disperse the fragrance through expelled air.
